(19) United States
(12) Patent Application Publication
(10) Pub. No.: US 2010/0044211 A1
(43) Pub. Date: Feb. 25, 2010

(54) APPARATUS AND METHOD FOR DETECTING TARGET MOLECULES

(76) Inventors: Sunghoon Kwon, Seoul (KR); Junhoi Kim, Seoul (KR)

Correspondence Address: Sunghoon Kwoon;Faculty Apt 122I-10, San 4-2, Bongchun 7 Dong, Gwanak-Gu, Seoul, KR

(21) Appl. No.: 12/197,867

(22) Filed: Aug. 25, 2008

(51) Int. Cl. G01N 33/483

(52) U.S. Cl. 204/165; 204/403.01; 427/125

(57) ABSTRACT

The present technology provides apparatuses for the detection of one or more target molecules. The apparatuses include a membrane having a nanochannel configured to allow passage of the target molecule, an electrical detection unit, and an optical detection unit. The apparatuses are capable of detecting the location of one or more target molecules, the time at which the molecules arrive at the location, as well as the identity of the molecules. Also disclosed are methods of making the apparatuses and methods of using the apparatuses to detect target molecules, including single biomolecules.

### BACKGROUND

Techniques for the optical detection of molecules may be used to develop molecular sensors. Single molecule based sensors need to exceed the performance limit of existing molecular sensors, especially in DNA sequencing. Among various optical single molecule detection techniques, surface-enhanced Raman scattering spectroscopy (SERS) offers the unique advantage of label-less detection capability.

### SUMMARY

In one embodiment, an apparatus for detecting one or more target molecules comprises a membrane separating a first chamber and a second chamber, the membrane comprising a nanochannel configured to allow passage of the one or more target molecules, an electrical detection unit configured to detect the passage of the one or more target molecules through the nanochannel, and an optical detection unit configured to identify the one or more target molecules passing through the nanochannel. Each of the first and second chambers may comprise an electrolyte. The membrane may comprise a dielectric material. The dielectric material may comprise silicon nitride, silicon oxide, glass, titanium oxide, tantalum oxide, aluminum oxide, or quartz. The membrane may comprise a Raman scattering enhancing material disposed on a surface of the membrane. The Raman scattering enhancing material may comprise one or more metals. The metals may be selected from gold, silver, platinum, copper, or aluminum. The nanochannel may have a diameter of about 20 nm or less. The electrical detection unit may be configured to apply a voltage or current across the membrane, and to detect a current signal change upon passage of the one or more target molecules through the nanochannel. The optical detection unit may be configured to apply an electromagnetic energy source to the one or more target molecules and to detect an optical signal from the one or more target molecules generated by the source. The optical signal may be Raman scattering or fluorescence. The electromagnetic energy may comprise visible light, infrared ray, X-ray, or UV ray. The one or more target molecules may comprise one or more biomolecules. The one or more biomolecules may comprise polypeptide, DNA, RNA, or protein molecules. The one or more target molecules may comprise one or more fluorescent tags.

In another embodiment, a method of detecting one or more target molecules comprises applying an electrical source across a membrane comprising a nanochannel configured to allow passage of the one or more target molecules, detecting an electrical signal change upon passage of the one or more target molecules through the nanochannel, applying an electromagnetic energy source to the one or more target molecules, and detecting an optical signal from the one or more target molecules generated by the electromagnetic energy source. The electrical source may comprise a voltage or a current source, and the electrical signal change may comprise a current signal change. The optical signal may be Raman scattering or fluorescence, and the membrane may comprise a Raman scattering enhancing material disposed on a surface of the membrane. The Raman scattering enhancing material may comprise one or more metals, which may be selected from gold, silver, platinum, copper or aluminum.

In another embodiment, a method of manufacturing an apparatus for detecting one or more target molecules comprises providing a system comprising a membrane separating a first chamber and a second chamber, the membrane comprising a nanochannel configured to allow passage of the one or more target molecules, providing an electrical detection unit configured to detect the passage of the one or more target molecules through the nanochannel, and providing an optical detection unit configured to identify the one or more target molecules passing through the nanochannel. A layer of a Raman scattering enhancing material may be formed on the surface of the membrane, and may comprise one or more metals selected from gold, silver, platinum, copper or aluminum. The optical detection unit may be configured to apply an electromagnetic energy source to the one or more target molecules and to detect an optical signal from the one or more target molecules generated by the source, and the optical signal may be Raman scattering or fluorescence. The electrical detection unit may be configured to apply a voltage or current across the membrane and to detect a current signal change upon passage of the one or more target molecules through the nanochannel.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1 and 2 depict illustrative embodiments of a molecular target detection apparatus.

FIGS. 3a and 3b depict illustrative embodiments of a portion of a molecular target detection apparatus, including the membrane.

FIG. 4 depicts an illustrative embodiment of a graph illustrating an electrical signal change detected by a molecular target detection apparatus.

FIG. 5 depicts an illustrative embodiment of a graph illustrating an optical signal detected by a molecular target detection apparatus.

FIG. 6 shows a flow chart of illustrative embodiment of a method of detecting one or more target molecules.

### DETAILED DESCRIPTION

In the following detailed description, reference is made to the accompanying drawing, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Disclosed herein are apparatuses capable of detecting the location of one or more target molecules, the time at which the molecules arrive at the location, as well as the identity of the molecules. Thus, the apparatuses allow for the multimodal detection of target molecules, including single molecules. Also disclosed are methods of using and manufacturing the apparatuses.

Hereinafter, target detection apparatuses and target detection methods according to illustrative embodiments will be described in detail with reference to the attached drawings.

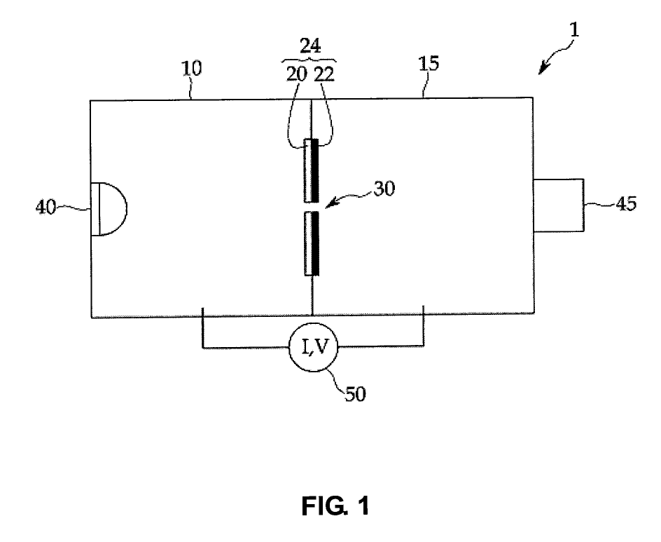

FIG. 1 illustrates an apparatus 1 for detecting one or more target molecules according to an example embodiment. In one embodiment, the molecular target detection apparatus 1 includes first and second chambers 10, 15 that are separated from each other by a membrane 24. The first and second chambers 10, 15 may be filled with an electrolyte. By way of example only, the electrolyte may be an aqueous buffer solution. The aqueous buffer solution may be prepared by any method known in the art. By way of example only, the solution may be prepared by mixing 1 M KCl, 10 mM Tris-HCl, pH 8.0, and 1 mM EDTA. One or more target molecules are introduced to the first chamber 10. The target molecules may be the same or different from each other. The target molecules may include biomolecules. Exemplary biomolecules include, but are not limited to polypeptide, DNA, RNA, or protein molecules. By way of example only, biomolecules can include single-stranded DNA (ssDNA), double-stranded DNAs (dsDNAs), cDNAs, mRNAs, rRNAs, oligonucleotides, peptides, antigens, antibodies (e.g., monoclonal or polyclonal), aptamers, and/or any natural and/or non-natural modifications or derivatives thereof.

As illustrated, the membrane 20 that is disposed between the first and second chambers 10, 15 includes a nanochannel or nanohole 30 penetrating the membrane 20. The size of the nanochannel 30 may be adjusted depending on the size (for example, an average diameter) of a target molecule to be detected. The nanochannel 30 may be formed to have a small feature size. The size of the nanochannel 30 can refer to an average diameter. The size of the nanochannel 30 is about 100 nm or less, particularly about 50 nm or less, more particularly about 20 nm or less.

The membrane 20 is provided to separate the first and second chambers 10, 15. In some embodiments, the membrane 20 may be formed of a dielectric material or an insulating material. In some embodiments, the material of the membrane 20 may include, but not limited to, silicon oxide, silicon nitride, glass, titanium oxide (TiO₂), tantalum oxide (Ta₂O₅), aluminum oxide (Al₂O₅), quartz, etc.

The membrane 20 may further comprise a Raman scattering enhancing material 22 disposed on a surface of the membrane 20 facing the second chamber 15. The Raman scattering enhancing material 22 can include one or more metals. In some embodiments, the Raman scattering enhancing material 22 may include metals having the optical characteristics of surface plasmon resonance, such as, but not limited to, gold, silver, platinum, copper, and aluminum.

The Raman scattering enhancing material 22 enhances Raman scattering and thus, upon illumination of the target molecule, the Raman scattering of the target molecule is enhanced, facilitating the detection of the optical signal from the target molecule. Hereinafter, the membrane 20 coated with the Raman scattering enhancing material 22 is indicated as a metal coated membrane 24 in some embodiments.

The metal-coated membrane 24 may have a thickness of about 500 nm or less, particularly about 100 nm or less, more particularly about 10 nm to 50 nm.

The metal coated-membrane 24 can be manufactured by forming a dielectric layer, forming a metal layer on the dielectric layer using known metal forming methods (e.g., evaporation), and forming a nanochannel penetrating the dielectric layer and the metal layer using known lithography and etching methods (e.g., focused ion-beam lithography, e-beam lithography or extreme-UV lithography). Another methods for manufacturing the metal coated membrane 24 can be used and above-described method is just one example.

An electrical detection unit 50 can comprise an electrical source and an electrical detector to detect the disclosed electrical signal change. An electrical source is provided between the first and second chambers 10, 15. The electrical source may apply a voltage between the first and second chambers 10, 15 to have a charged target pulled toward the nanochannel 30 of the membrane 24. The electrical source may be, for example, a voltage or current source. The electrical detector may be configured to detect a current signal change. The electrical detector may detect a current signal change when a target blocks the nanochannel 30 and thus an electrical current flowing through the nanochannel 30 is hindered. In one embodiment, the electrical detection unit 50 may comprise an electrical processor to process, analyze, store, or transmit the electrical signals detected by the electrical detector.

An optical detection unit is configured to apply an electromagnetic energy to the one or more target molecules and to detect an optical signal from the one or more target molecules. The optical detection unit may comprise an electromagnetic energy source 40 and an optical detector 45 to detect the optical signals from the target molecules. The electromagnetic energy source 40 for applying an electromagnetic energy to a target in the vicinity of the membrane 24 can be provided to the first chamber. In some embodiments, the electromagnetic energy comprises, but not limited to, visible light, infrared ray, X-ray, or UV ray. As illustrated, the electromagnetic energy source 40 may be provided within the first chamber 10. In another embodiment, the electromagnetic energy source 40 may be provided outside the first chamber 10 in such a manner that it can apply an electromagnetic energy into the first chamber 10. By way of example only, the electromagnetic energy source 40 such as light source illuminates light onto a target molecule in the vicinity of the nanochannel 30 in order to obtain the optical characteristics of the target molecule. The electromagnetic energy source 40 may be configured to apply an electromagnetic energy to the target at least when the electrical detection unit detects an electrical signal change according to the movement of the target. In one embodiment, the electromagnetic energy source 40 may be configured to apply an electromagnetic energy to the target in response to the electrical signal change detected by the electrical detection unit. In another embodiment, the electromagnetic energy source 40 may provide the electromagnetic energy to the target molecule for a predetermined time until the optical characteristics of the target molecule is obtained.

The optical detector 45 can be provided to the second chamber 15. As illustrated, the optical detector 45 may be provided outside the second chamber 15, and observe optical phenomena in the second chamber 15, but the configuration of the optical detector 45 is not limited thereto. The optical detector 45 may detect fluorescence or the Raman scattering signal of the target molecule. The optical detector 45 may include, but not limited to, an optical microscope or a confocal microscope. In some embodiments, the optical detector 45 may include a processor (not illustrated) for processing, analyzing, storing, or transmitting the optical information of a target included in a sample. Alternatively, a processor may be provided independently from the optical detector 45. In some embodiments, the processor may be connected to the optical detector 45 and can process, analyze, store or transmit the optical information detected by the optical detector 45. The processor may include a computer.

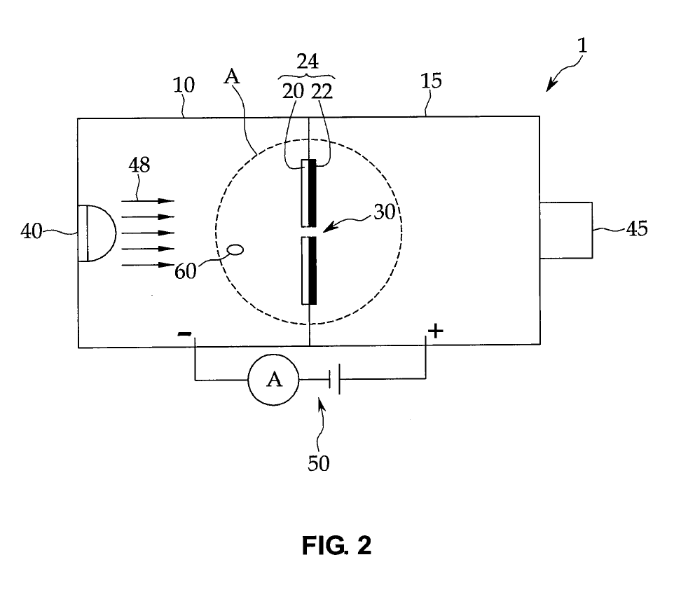

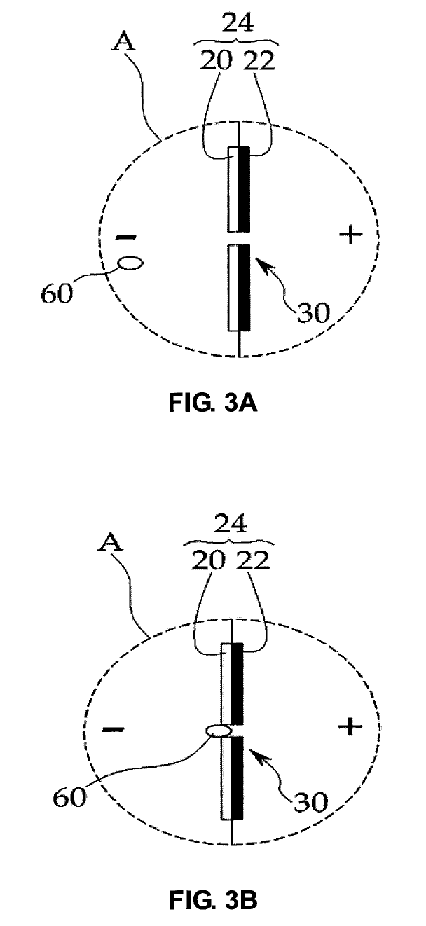

Now, a method of detecting one or more target molecules, by way of example only, a single molecule by using the above-mentioned apparatus 1 will be described, with reference to FIGS. 2, 3a and 3b. FIG. 2 shows an illustrative embodiment of a method of detecting one or more target molecules by using the apparatus 1 shown in FIG. 1. FIGS. 3a and 3b each illustrate an enlarged view of the part designated by “A” in FIG. 2.

Referring to FIG. 2, the apparatus 1 includes first and second chambers 10, 15 separated from each other by a membrane 24, an electromagnetic energy source 40 provided on one side of the first chamber 10, an optical detector 45 provided on one side of the second chamber 15, and an electrical detection unit 50 for applying an electrical source between the first and second chambers 10, 15 and detecting an electrical signal, as described above.

The electrical detection unit 50 applies an electrical source across the membrane 24 comprising a nanochannel 30 configured to allow passage of the target molecule 60. The electrical detection unit 50 may include an electrical source (e.g. voltage source 50b) for applying a voltage between the first and second chambers 10, 15 and an electrical detector (e.g. a current detector 50a) for detecting a current signal change.

An electrolyte is filled in the first and second chambers 10, 15, and a target molecule (for example, a single molecule) 60 is supplied into the first chamber 10. In response to the voltage applied between the first and second chambers 10, 15, the anions within the electrolyte move to the second chamber 15 of a positive pole (+), and the cations within the electrolyte move to the first chamber 10 of a negative pole (−). Then, the target 60 of negative polarity (−) is pulled toward the nanochannel 30 of the membrane 24. If the target 60 has a positive polarity (+), the electrical source applies the opposite voltage such that the second chamber 15 presents a negative pole (−), and the first chamber 10 presents a positive pole (+).

In one embodiment, if a voltage is applied across the membrane 24 having the nanochannel 30, the target molecule 60 supplied into the first chamber 10 (FIG. 3a) is pulled toward the nanochannel 30 of the membrane 24 and then blocks the nanochannel 30 (FIG. 3b). When the target molecule 60 blocks the nanochannel 30, an electrical current flowing through the nanochannel 30 is changed. Therefore, the electrical signal (e.g. current signal) change upon passage of the target molecule 60 through the nanochannel 30 is detected by the electrical detector.

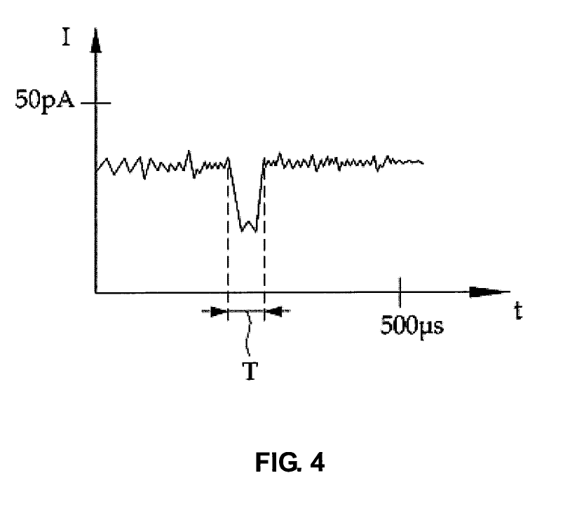

FIG. 4 depicts an illustrative embodiment of a graph showing an electrical signal change in a molecular target detection method according to an example embodiment. In the graph the interval T at the time axis (t) represents a period of time that a target molecule 60 resides in the nanochannel 30 and hinders an electrical current flowing through the nanochannel 30. As shown in the graph, the magnitude I of a current signal is significantly reduced during the interval T. Thus, by detecting a current signal change, the time the target molecule 60 is located within or near the vicinity of the nanochannel 30 can be detected.

The optical detection for a target will be described with reference to FIG. 2 again. The electromagnetic energy source 40 generates an electromagnetic energy capable of activating Raman scattering of a target and applies the electromagnetic energy to the target. The electromagnetic energy comprises, but not limited to, visible light, infrared ray, X-ray, or UV ray. By way of example only, the light 42 from the light source 40 illuminates the membrane 24 including the nanochannel 30. The light 42 is transmitted to the membrane surface facing the second chamber 15, by way of example only, a Raman scattering enhancing material coating 22. The photons of the light are converted into surface plasmons at the boundary surface between the membrane 20 and the Raman scattering enhancing material coating 22 of the membrane 24. Then, the surface plasmons are converted into photons again on the surface of the Raman scattering enhancing material coating 22. The surface plasmons can be detected by the optical detector 45. Light transmission may be enhanced around the nanochannel 30. Also, the Raman scattering enhancing material coating 22 can enhance the excitement of surface plasmons.

When a target molecule 60 is located within or in the vicinity of the nanochannel 30 or passes through the nanochannel 30, a Raman scattering signal corresponding to the optical characteristics of the target 60 is detected by the optical detector 45. The Raman scattering signal can be detected in a spectrum. The identity of the target molecule may be determined by comparing the detected spectrum of the Raman scattering signal and spectra of known molecules. Thus, each target molecule in a mixture of different target molecules can be identified by its characteristic Raman scattering spectrum.

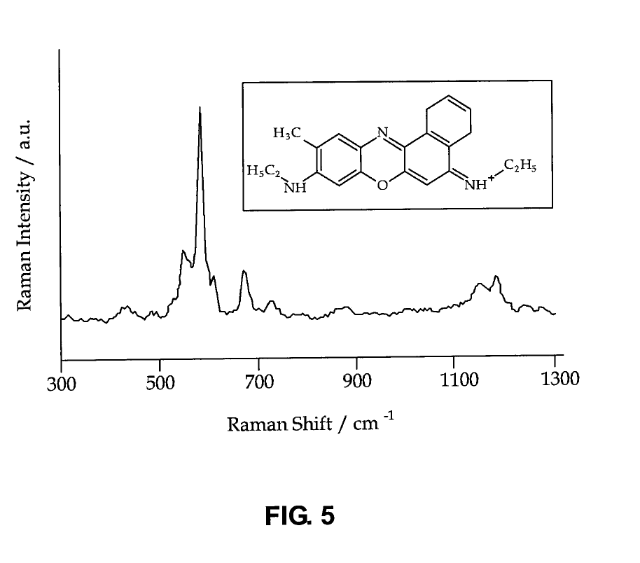

FIG. 5 depicts an illustrative embodiment of a graph showing a Raman signal in a molecular target detection method according to an example embodiment. In FIG. 5, spectrum of Raman scattering from oxazine 720 (oxa) is illustrated as an example. A common laser dye with an absorption band at ca. 620 nm was used. As such, each target molecule even in a mixture of different target molecules can be identified by its unique characteristic of Raman scattering spectrum.

In another example embodiment, the optical detector 45 may detect a target 60 with a fluorescent tag attached to the target 60, instead of detecting the Raman scattering signal of the target molecule 60. Fluorescent tags may include, but are not limited to, fluorescein and green fluorescent protein. Such tags may be coupled to the target molecules by well-known synthetic techniques.

According to the present technologies, a multimodal detection is possible for the molecular target detection. The first mode detection can be performed by the electrical detection unit to detect when a target 60 passes through the nanochannel 30. The second mode detection can be performed to detect the identity of the target by the optical detection unit including the electromagnetic energy source 40 and the optical detector 45. The second mode detection detects the optical characteristics of the target molecule, such as the Raman scattering signal or fluorescence of the target molecule.

Accordingly, the molecular target detection according to the embodiment can precisely detect when a target molecule such as a single molecule or a biomolecule passes through the nanochannel 30, while simultaneously determining the identity of the target molecule.

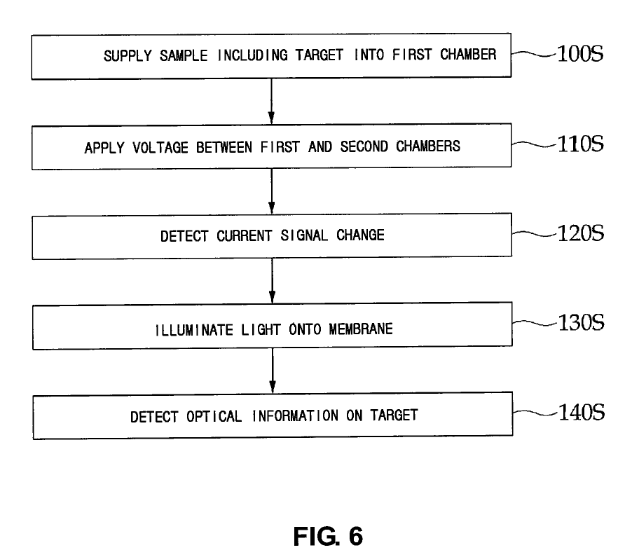

FIG. 6 is a flow chart showing an illustrative embodiment of a method of detecting one or more target molecules by using the molecular target detection apparatus.

Referring to FIG. 6, a sample including one or more target molecules is supplied into the first chamber 10 (100S). Next, an electrical source is applied across the membrane 24 having the nanochannel 30 configured to allow passage of the target molecule (110S), and an electrical signal change is detected upon passage of the target molecule through the nanochannel (120S). The electrical source may be, by way of example only, a voltage source. In one embodiment, if the target molecule blocks the nanochannel 30 or resides in the nanochannel 30, an electrical current flowing through the nanochannel 30 is hindered by the target molecule. As a result, a reduced current signal can be detected. Then, an electromagnetic energy such as, but not limited to, visible light, infrared ray, X-ray, or UV ray is radiated onto the membrane 24 (130S), and the optical signal of the target molecule located around the nanochannel 30 or passing through the nanochannel 30 is obtained (140S). In some embodiments, the optical signal may include Raman scattering or fluorescence, and the target molecule may be identified based on the optical information. In one embodiment, in response to the electrical signal change, an electromagnetic energy source can be radiated onto the target molecule. In some embodiments, the electromagnetic energy source can be provided to the target molecule for a certain period time until the optical signal of the target molecule is obtained, or constant source of illumination can be provided to the target molecule.

In another embodiment, a method of manufacturing an apparatus for detecting one or more target molecules described in FIG. 1 is provided.

First of all, a system comprising a membrane 24 separating a first chamber 10 and a second chamber 15 is provided. The membrane 24 comprises a nanochannel 30 configured to allow passage of the one or more target molecules. An electrical detection unit 50 configured to detect the passage of the one or more target molecules through the nanochannel 30 is provided. The electrical detection unit 50 may include an electrical source to apply an electrical source (e.g., voltage source) between the first and second chambers 10, 15 and an electrical detector to detect a current signal change, which is caused when a target molecule supplied into the first chamber 10 blocks the nanochannel 30, thereby hindering the electrical current flowing through the nanochannel 30. An optical detection unit configured to identify the one or more target molecules passing through the nanochannel is provided. The optical detection unit can comprise an electromagnetic energy source 40 provided to the first chamber 10 to provide radiation of the electromagnetic energy and an optical detector 45 provided to the second chamber 15 to detect the optical signal from the one or more target molecules generated by the electromagnetic energy source.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An apparatus for detecting one or more target molecules, the apparatus comprising:
a membrane separating a first chamber and a second chamber, the membrane comprising a nanochannel configured to allow passage of the one or more target molecules;
an electrical detection unit configured to detect the passage of the one or more target molecules through the nanochannel; and
an optical detection unit configured to identify the one or more target molecules passing through the nanochannel.

2. The apparatus of claim 1, wherein each of the first and second chambers comprises an electrolyte.

3. The apparatus of claim 1, wherein the membrane comprises a dielectric material.

4. The apparatus of claim 3, wherein the dielectric material comprises silicon nitride, silicon oxide, glass, titanium oxide, tantalum oxide, aluminum oxide, or quartz.

5. The apparatus of claim 1, wherein the membrane comprises a Raman scattering enhancing material disposed on a surface of the membrane.

6. The apparatus of claim 5, wherein the Raman scattering enhancing material comprises one or more metals.

7. The apparatus of claim 6, wherein the metals are selected from gold, silver, platinum, copper, or aluminum.

8. The apparatus of claim 1, wherein the nanochannel has a diameter of about 20 nm or less.

9. The apparatus of claim 1, wherein the electrical detection unit is configured to apply a voltage or current across the membrane, and to detect a current signal change upon passage of the one or more target molecules through the nanochannel.

10. The apparatus of claim 1, wherein the optical detection unit is configured to apply an electromagnetic energy source to the one or more target molecules and to detect an optical signal from the one or more target molecules generated by the source.

11. The apparatus of claim 10, wherein the optical signal is Raman scattering or fluorescence.

12. The apparatus of claim 10, wherein the electromagnetic energy comprises visible light, infrared ray, X-ray, or UV ray.

13. The apparatus of claim 1, wherein the one or more target molecules comprise one or more biomolecules.

14. The apparatus of claim 13, wherein the one or more biomolecules comprise polypeptide, DNA, RNA, or protein molecules.

15. The apparatus of claim 1, wherein the one or more target molecules comprise one or more fluorescent tags.

16. A method of detecting one or more target molecules, the method comprising:
applying an electrical source across a membrane comprising a nanochannel configured to allow passage of the one or more target molecules;
detecting an electrical signal change upon passage of the one or more target molecules through the nanochannel;
applying an electromagnetic energy source to the one or more target molecules; and
detecting an optical signal from the one or more target molecules generated by the electromagnetic energy source.

17. The method of claim 16, wherein the electrical source comprises a voltage or a current source, and the electrical signal change comprises a current signal change.

18. The method of claim 16, wherein the optical signal is Raman scattering or fluorescence.

19. The method of claim 16, wherein the membrane comprises a Raman scattering enhancing material disposed on a surface of the membrane.

20. The method of claim 19, wherein the Raman scattering enhancing material comprises one or more metals.

21. The method of claim 20, wherein the metals are selected from gold, silver, platinum, copper, or aluminum.

22. A method of manufacturing an apparatus for detecting one or more target molecules, the method comprising:
providing a system comprising a membrane separating a first chamber and a second chamber, the membrane comprising a nanochannel configured to allow passage of the one or more target molecules;
providing an electrical detection unit configured to detect the passage of the one or more target molecules through the nanochannel; and
providing an optical detection unit configured to identify the one or more target molecules passing through the nanochannel.

23. The method of claim 22, further comprising forming a layer of a Raman scattering enhancing material on a surface of the membrane.

24. The method of claim 23, wherein the Raman scattering enhancing layer comprises one or more metals, the metals selected from gold, silver, platinum, copper, or aluminum.

25. The method of claim 22, wherein the optical detection unit is configured to apply an electromagnetic energy source to the one or more target molecules and to detect an optical signal from the one or more target molecules generated by the source.

26. The method of claim 25, wherein the optical signal is Raman scattering or fluorescence.

27. The method of claim 22, wherein the electrical detection unit is configured to apply a voltage or current across the membrane and to detect a current signal change upon passage of the one or more target molecules through the nanochannel.
